(19) United States
(12) Patent Application Publication
(10) Pub. No.: US 2005/0130986 A1
(43) Pub. Date: Jun. 16, 2005

(54) Treatment of spondylarthropathies

(76) Inventor: Kari Kalervo Eklund, Helsinki (FI)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/985,441

(22) Filed: Nov. 10, 2004

Related U.S. Application Data
(60) Provisional application No. 60/524,228, filed on Nov. 21, 2003.

(51) Int. Cl. A61K 31/506

(52) U.S. Cl. 514/252.18

(57) ABSTRACT

4-(4-methylpiperazin-1-ylmethyl)-N-[4-methyl-3-(4-pyridin-3-yl)pyrimidin-2-ylamino)phenyl]-benzamide of the formula I 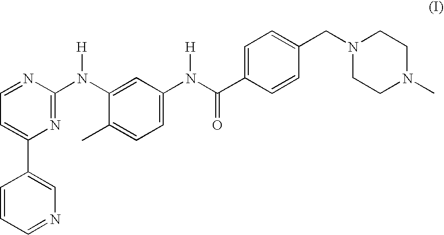 or a pharmaceutically acceptable salt thereof can be used in the treatment of spondylarthropathies. The invention also relates to a combination of the compound of the formula I or a pharmaceutically acceptable salt thereof with one or more disease modifying drugs selected from a group consisting of DMARDs.

The invention relates to the use of 4-(4-methylpiperazin-1-ylmethyl)-N-[4-methyl-3-(4-pyridin-3-yl)pyrimidin-2-ylamino)phenyl]-benzamide, hereinafter referred as “COMPOUND I”, or a pharmaceutically acceptable salt thereof for the manufacture of pharmaceutical compositions for use in the treatment of spondylarthropathies, e.g. ankylosing spondylitis or sporiatic arthritis, to the use of COMPOUND I or a pharmaceutically acceptable salt thereof in the treatment of spondylarthropathies, to a method of treating warm-blooded animals including mammals, e.g. humans, suffering from or susceptible to spondylarthropathies by administering to a said animal in need of such treatment an effective dose of COMPOUND I or a pharmaceutically acceptable salt thereof.

The invention also relates to a combination of COMPOUND I or a pharmaceutically acceptable salt thereof with one or more disease modifying arthritis rheumatoid drugs abbreviated as DMARDs, to treat warm-blooded animals including mammals, especially humans having or susceptible to spondylarthropathies.

The term “spondylarthorpathy or spondylarthropathies” as used herein especially relates to the disease of a patient which fulfils the criteria for spondylarthorpathy as determined by the ESSG criteria, the AMOR criteria or both, ankylosing spondylitis, psoriatic arthropathy, undifferentiated spondyloarthropathy, reactive arthritis, Reiters syndrome, arthropathy associated with inflammatory bowl diseases, SAPHO syndrome or Morbus Bechet.

Spondylarthropathies, also called spondyloarthropathies, are a class of inflammatory diseases the etiology of which is unknown. Spondylarthropathies comprise a group of diseases for which certain symptoms or signs are common. Spondylarthropathies are generally characterized by chronic inflammation involving, e.g. the sacroiliac joints, peripheral joints, the entheses, i.e. insertion site of the tendons, and axial skeleton. Not all patients have inflammation of the spine. Other symptoms can occur as dactylitis, i.e. the whole finger or the toe swells and inflammation of the eye called uveitis. Chronic inflammation of entheses, called enthesitis, can lead to new bone formation, syndesmophytes and ankylosis of joints primarily in the axial skeleton. Chronic inflammation leads typically to the formation of syndesmophytes in the spine and erosions and ankylosis of the sacroiliac joints and in peripheral joints the result can be severe erosive destruction of the joints. The different types of spondylarthropathies and their clinical presentations are familiar to one of skill in the art.

### BACKGROUND INFORMATION

The spondylatrthropathy is considered to have 5 subtypes which are ankylosing spondylitis, psoriatic arthritis also called psoriasis arthropathy, undifferentiated spondylarthropathy, reactive arthritis, Reiters syndrome, and arthropathies associated with inflammatory bowel diseases such as Crohn's disease and Colitis ulcerosa. SAPHO syndrome is a subgroup of psoriasis arthropathy and is considered to belong to the spondylarthropathies. Morbus Bechet can be considered to belong to the group of spondyloarthropathies in the present context.

Ankylosing spondylitis is the classical arthropathy disease. Typical symptoms are sacroilitis, inflammation of the spine leading into ankylosis of the spine. Inflammation of the peripheral large and/or small joints is typical. A typical feature can be enthesitis. It is associated with the HLA-B27 factor which is an inherited gene. Uveitis is relatively common.

Undifferentiated spondyloarthropathy is like the classical ankylosing spondylitis. It is associated with the HLA-B27, typical symptoms include sacroilitis; inflammation of the large joints, enthesitis but it usually does not have the inflammation of the spine as in the classical ankylosing spondylitis.

Reactive arthritis/Reiters syndrome is a spondylarthropathy disease one can get after certain infections which include gastroenteritis with e.g. salmonella, yersinia, shigella, and after certain sexually transmitted diseases, e.g. chlamydia. Most cases are self-limited and last about 3-4 months but it can become chronic and then it is very difficult to treat. Reactive arthritis and Reiters syndrome are similar to ankylosing spondyitis and undiffentiated spondylarthropathy. When there is in addition to joint symptoms rash and certain other symptoms it is called Reiters syndrome.

Psoriasis arthropathy is associated with typical spondyloarthropathy type joint symptoms when they are associated with psoriasis rash. Typical features can include sacroilitis and inflammation of the spine, but not all patients have inflammation of the spine. Enthesitis is a common feature of psoriasis arthropathy as well as symptoms and inflammation of finger joints. Up to 40% of patients with psoriasis have also arthropathy.

Arthropathy associated with inflammatory bowl diseases, e.g. in Crohns disease and in colitis ulcerosa there can be joint symptoms which belong to the group of spondyloarthropathy. Again sacroilitis, enthesitis, inflammation of the large joints are typical but inflammation of small joint can occur.

Juvenile form spondyloarthropathy affects children and is very similar to adult form.

There are diagnostic criteria developed for spondyloarthropathy, e.g. the European Spondyloarthropathy Study Group preliminary criteria for classification of spondyloarthropathy abbreviated as ESSG criteria, see for example Dougados et al., The. Arthritis Rheumatism 1991, 34:1218-27 or the Amor criteria for spondylarthropathies, see the following reference Amor B. et al., Ann. Med. Interne (Paris) 1991, 142(2): 85-9. The ESSG criteria is the most commonly used criteria to identify spondylarthropathy patients.

Spondyloarthropathy, as a group of diseases, is not related to rheumatoid arthritis although both groups of patients do have joint symptoms. Spondylarthropathies are pathogenetically clearly different from rheumatoid arthritis. Many of the spondylarthropathies are associated with the HLA-B27 gene but psoriasis arthritis and arthropathies in inflammatory bowl diseases are not. If one patient has rheumatoid arthritis his/her chances of having spondyloarthropathy is about as great as if she/he did not have rheumatoid arthritis. Having both diseases is a uncommon coincidence.

The prevalence of spondyloarthropathy, as a group of diseases, has been estimated to be as high as 0.6-1.9%, see for example Brandt J et al. Rheumatology 1999; 38: 831-36.

Spondylarthropathies are associated with significant morbidity, work disability and increased mortality. The treatment possibilities for spondylarthropathies are currently very limited with conventional therapeutics having only limited usefulness. Non steroidal anti-inflammatory drug, abbreviated as NSAIDs, have been used and then DMARDs for the treatment of spondylarthropathy. The scientific proof that the DMARDs would work in treating spondylarthropathy is very poor and in many trials no efficacy has been observed. There is minor efficacy observed with sulfasalazine and with methotrexate mainly in the treatment of peripheral arthritis. In sporiasis arthropathy, methotrexate has shown some efficacy. Thus, there is need for an efficient treatment for spondylarthropathy.

### DESCRIPTION OF THE INVENTION

It has now surprisingly been demonstrated that spondylarthropathies can be successfully treated with COMPOUND I or a pharmaceutically acceptable salt thereof, e.g. the monomethane sulfonate salt of COMPOUND I hereinafter called SALT I. COMPOUND I is 4-(4-methylpiperazin-1-ylmethyl)-N-[4-methyl-3-(4-pyridin-3-yl)pyrimidin-2-ylamino)phenyl]-benzamide having the formula I

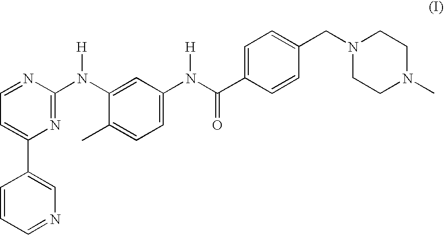

The preparation of the 4-(4-methylpiperazin-1-ylmethyl)-N-[4-methyl-3-(4-pyridin-3-yl)pyrimidin-2-ylamino)phenyl]-benzamide is described in the EP-A-0 564 409. The monomethanesulfonic acid addition salt of COMPOUND I and crystal forms thereof, e.g. the alpha crystal form and the beta crystal form, are described in PCT patent application WO99/03854 published on Jan. 28, 1999. The pharmaceutical tablet comprising Compound I or a pharmaceutically acceptable salt thereof is described in the PCT patent application WO03/090720 published on Nov. 6, 2003.

Pharmaceutically acceptable salts of COMPOUND I are pharmaceutically acceptable acid addition salts, for example, with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid.

The invention relates to the use of COMPOUND I or a pharmaceutically acceptable salt thereof, for the manufacture of pharmaceutical compositions for the treatment of spondylarthropathy, e.g. ankylosing spondylitis or psoriatic arthropathy.

The present invention relates to the use of Compound I or a pharmaceutically acceptable salt thereof, for the manufacture of pharmaceutical compositions for the treatment of the disease of a patient which fulfils the criteria for spondylarthorpathy as determined by the ESSG criteria, the AMOR criteria or both, ankylosing spondylitis, psoriatic arthropathy, undifferentiated spondyloarthropathy, reactive arthritis, Reiters syndrome, arthropathy associated with inflammatory bowl diseases, SAPHO syndrome or Morbus Bechet.

The present invention pertains to the use of COMPOUND I or a pharmaceutically acceptable salt thereof for the manufacture of a medicament for the treatment of ankylosing spondylitis.

The present invention pertains to the use of COMPOUND I or a pharmaceutically acceptable salt thereof for the manufacture of a medicament for the treatment of psoriatic arthropathy.

In one embodiment, the invention relates to the use of COMPOUND I or a pharmaceutically acceptable salt thereof for the manufacture of a medicament for the treatment of the disease of a patient which fulfils the criteria for spondylarthorpathy as determined by the ESSG criteria, the AMOR criteria or both.

In one embodiment, the invention relates to the use of a pharmaceutically acceptable acid addition of COMPOUND I, e.g. the monomethanesulfonate salt, e.g. the alpha crystal form of the monomethane sulfonate salt or the beta crystal form of said salt or mixture thereof, for the manufacture of pharmaceutical compositions for the treatment of spondylarthropathy, e.g. ankylosing spondylitis or psoriatic arthropathy.

According to one embodiment of the invention, SALT I is administered.

The dosages are expressed as the dose of COMPOUND I free base administered, e.g. for a 100 mg dose, 119.5 mg of SALT I is administered corresponding to 100 mg of COMPOUND I free base. For example, a dose of 400 mg of COMPOUND I has to be understood as 478 mg SALT I being administered corresponding to 400 mg of COMPOUND I free base.

Depending on species, age, individual condition, mode of administration, and the clinical picture in question, effective doses, for example, daily doses corresponding to an amount of about 100-1000 mg, e.g. 200 to 800 mg, preferably 200-600 mg, especially 400 mg of COMPOUND I free base, are administered to warm-blooded animals of about 70 kg bodyweight. For adult patients with spondylarthropathy, e.g. ankylosing spondylitis, a starting dose of 200-400 mg daily can be recommended. For patients with an inadequate response after an assessment of response to therapy with 400 mg daily, dose escalation can be safely considered and patients may be treated as long as they benefit from treatment and in the absence of limiting toxicities. In this way, for example, one of skill in the art may determine an effective dose of COMPOUND, I or a pharmaceutically acceptable salt thereof to be administered to the patient.

The invention relates also to a method for administering to a human subject suffering from a spondylarthropathy, e.g. ankylosing spondylitis or psoriatic arthropathy, COMPOUND I or a pharmaceutically acceptable salt thereof, which comprises administering a pharmaceutically effective amount of COMPOUND I or a pharmaceutically acceptable salt thereof. The invention relates especially to such method wherein a daily dose of 100 to 1000 mg, 200 to 800 mg, especially 400-600 mg, e.g. 400 mg of COMPOUND I, e.g. in the form of SALT I, is administered.

The invention relates also to said method for administering to a human subject suffering from spondylarthropathy, e.g. ankylosing spondylitis or psoriatic arthropathy, COMPOUND I or a pharmaceutically acceptable salt thereof, which comprises administering a pharmaceutically effective amount of COMPOUND I or a pharmaceutically acceptable salt thereof to the human subject once daily for a period exceeding 3 months.

The invention also relates in a combination which comprises (a) COMPOUND I or a pharmaceutically acceptable salt thereof and (b) a therapeutic agent selected from a group consisting of DMARDs, most preferably a combination wherein the combination partners are present in synergistically effective amounts.

The DMARDs, some of which have been used as anti-spondylarthropathy drugs include, but are not limited to, any one or more of the following:
(1) a non steroidal anti-inflammatory drug, abbreviated as NSAID, such as acetylsalicylic acid, ibuprofen, diclofenac, tenoxicam, naproxen or cyclooxygenase-2 inhibitors, e.g. celecoxib, rofecoxib, valdecoxib;
(2) an anti-inflammatory steroidal drug such as 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, maziprednone, medrysone, meprednisone, methylprednisolone, mometasone furoate, paramethasone, prednicarbate, prednisolone, prednisolone 25-diethylamino acetate, prednisolone sodium phosphate, prednisone, prednival, prednylidene, rimexolone, tixocortol, triamcinolone, triamcinolone acetonide, triamcinolone benetonide, triamcinolone hexacetonide;
(3) salazosulfapyridine, sulfasalazine;
(4) methotrexate;
(5) leflunomide;
(6) intramuscular gold, e.g. aurathiomalate;
(7) anti-tumor necrosis factor□ agents, e.g. etanercept, infliximab or adalimumab.

The structure of the active agents identified by e.g. code numbers, generic or trade names, may be taken from the actual edition of the standard compendium “The Merck Index” or from databases, e.g. Patents International (e.g. IMS World Publications), or the publications mentioned above and below. The corresponding content thereof is hereby incorporated by reference.

The invention relates to the use of a combination which comprises (a) COMPOUND I or a pharmaceutically acceptable salt thereof and (b) a therapeutic agent selected from the disease modifying arthritis rheumatoid drugs abbreviated as DMARDs for the manufacture of a medicament for the treatment of spondylarthropathies.

The inventions pertains especially to the use of a combination comprising (a) COMPOUND I or a pharmaceutically acceptable salt thereof and (b) a therapeutic agent selected from the DMARDs for the manufacture of a medicament for the treatment of the disease of a patient which fulfils the criteria for spondylarthorpathy as determined by the ESSG criteria, the AMOR criteria or both, ankylosing spondylitis, psoriatic arthropathy, undifferentiated spondyloarthropathy, reactive arthritis, Reiters syndrome, arthropathy associated with inflammatory bowl diseases, SAPHO syndrome, Morbus Bechet.

In one embodiment of the invention, the combination partner (b) is an anti-tumor necrosis a factor agent.

In a further embodiment of the invention the anti-tumor necrosis factor agent is selected from etanercept, adalimumab or infliximab, preferably etanercept or infliximab.

In another embodiment of the invention, the combination partner (b) is selected from methotrexate, cyclosporine, intramusclar Gold preparation, azathioprine, leflunomide and hydroxychloroquine.

The invention relates to the combination as disclosed above wherein the combination partner (b) is an anti-inflammatory steroidal drug, most preferably, prednisone.

The effective dosage of each of the combination partners employed in the combination may vary depending on a variety of factors including the particular combination of the pharmaceutical compound partners, the route of administration, the severity of the disease, the renal and hepatic functions of the patient. The molar ratio (a)/(b) of the combination partners is about 0.1 to 10, most preferably 0.3 to 3. The unit dosage form contains the monomethanesulfonate salt of 4-(4-methylpiperazin-1-ylmethyl)-N-[4-methyl-3-(4-pyridin-3-yl)pyrimidin-2-ylamino)phenyl]-benzamide of the formula I, most preferably in the beta crystal form at a dose corresponding to 20 to 200 mg, preferably 50 to 150 mg of COMPOUND I free base, e.g. in case of combination treatment.

If the combination partners are administered as a free combination, the combination partner (b) can be administered according to the instructions provided by the supplier.

The invention further pertains to a commercial package comprising a combination which comprises (a) COMPOUND I or a pharmaceutically acceptable salt thereof and (b) a therapeutic agent selected from the disease modifying arthritis rheumatoid drugs together with instructions for use in the treatment of spondylarthropathy.

### EXAMPLE 1

### Findings in a Patient with Severe Spondyloarthropathy

The patient is a male who has radiologically verified bilateral sacroilitis and disease of large joints. He is also positive for the HLA-B27 antigen. The patient fulfills the ESSG criteria and the Amor criteria for spondylarthorpathy. The disease has been severe and very refractory to previous treatment. The disease has led, for example, to destruction of the hip joints which have both been replaced by endoprostheses. He has also had other peripheral joint symptoms of large joints, for example, inflammation in knees, ankle joints and shoulders. In addition he has had symptoms also in small peripheral joints such as fingers and toes. He has also concomitant rheumatoid arthritis, and secondary Sjögrens syndrome with positive speckled antinuclear antibodies and positive rheumatoid factor. The previous anti-rheumatic medication has included methotrexate, cyclosporine, infliximab, intramusclar. Gold preparation, azathioprine, leflunomide, and they all have been used also in various combinations and have proved inefficient.

During the study the vital signs and blood chemistry was controlled first weekly, then biweekly and after three months once per month.

Administration of the Study Drug

Study medication: study medication, called the “study drug”, i.e. imatinib mesylate, is added to concomitant medication which included hydroxychloroquine 300 mg per day, prednisone 10 mg per day and ibuprofen 400-1600 mg per day. No changes in the doses of concomitant medication is allowed during the first three months of the study. No corticosteroid injections are given. Thereafter, the dose of corticosteroid was decreased due to the excellent therapeutic effect of the study drug. Study drug is given orally with a starting dose of 100 mg twice daily and increasing the dose after two weeks to 300 mg per day and then after 4 weeks to 400 mg per day which is continued thereafter.

Outcome Measures:

Swollen joint count, tender joint count, erythrocyte sedimentation rate, (ESR) and C-reactive protein (CRP), pain as measured using visual analog pain scale (VAS), disease activity as using visual analog scale and health assessment questionnaire are used as outcome measures. The antirheumatic effect could be observed already two weeks after the study drug was started as the movements in shoulder joints improved. Thereafter inflammation in ankles and knees resided and clear effect in swollen joint counts was observed after 2-3 months. He has continued to improve and after 18 months no serious side effects have been observed. Except for the slight dryness of the skin, the patient has not observed any significant side effects and he has felt fine. In addition no significant infections has occurred during the treatment.

All the outcome measures improved after two months of treatment with the study drug and continued to improve during the 18 month of treatment with the study drug. The outcome measures included swollen joint count which decreased from 31 before the treatment to 5 after the 18 month treatment; tender joint count decreased from 10 before the treatment to 1 after the treatment; pain experienced by the patient as estimated by the visual analogue scale (VAS) decreased from 37 mm before treatment to 11 mm after treatment; disease activity as estimated by the patient (VAS) decreased from 69 mm before treatment to 11 mm after treatment. Also the inflammatory parameters responded to the treatment. The CRP before the treatment was 42 mg/L and after the treatment 11 mg/L. The erythrocyte sedimentation rate before the treatment was 28 mm/h and after the treatment 11 mm/h. Finally the ability of the patient to cope in everyday life improved. This is illustrated by the health assessment questionnaire score (HAQ) which reflects the patients functional ability. High score indicates a high degree of disability. The HAQ score before the treatment is 1.5 and after 18 months of treatment it was decreased to 0.75.

### EXAMPLE 2

### Findings in a Patient with Psoriatic Arthritis

The patient is a male with established diagnosis of psoriasis arthropathy. He has had symptoms in back and spine and also in peripheral joints in knees, wrists, ankles, toes and in several finger joints for several years. He also has significant psoriasis rash. The previous anti-rheumatic therapy has included methotrexate at a dose of 20 mg per week and cyclosporine with the dose of 150 mg which both were discontinued because of lack of efficacy. Different preparations of non-steroidal anti-inflammatory compounds had been used alone and also concomitantly with methotrexate and cyclosporine without success.

During the 3 month study the vital signs and blood chemistry are controlled first weekly during the first month and then biweekly during the second month and then once per month.

Administration of the Study Drug

Study medication: study medication, called the “study drug”, i.e. COMPOUND I or a pharmaceutically acceptable salt thereof, e.g. imatinib mesylate or SALT I, is added to concomitant medication which included only non-steroidal anti-inflammatory drug naproxen with a dose of 500 mg twice per day. No changes in the doses of concomitant medication is allowed during the study. The knee joint and wrist joint are treated with corticosteroid injection during the first three weeks of the study but thereafter no other injections are given or needed. The injected joints are omitted from the analysis. Study drug is given orally with a starting dose of 100 mg twice daily and increasing the dose after two weeks to 300 mg per day and then after 4 weeks to 400 mg per day which is continued thereafter until the end of the study.

Outcome Measures

Swollen joint count, tender joint count, erythrocyte sedimentation rate, (ESR) and C-reactive protein (CRP), pain as measured using visual analog pain scale (VAS), disease activity as measured using VAS, night pain as measured using VAS, and the BASDAI (Bath Ankylosing Spondylitis Activity Index) score are used as outcome measures. All the outcome measures improve during the three month treatment. The positive effect of the treatment is observed after two months of treatment with the study drug and the efficacy is even more clearly evident after three months of the use of the study drug. The number of swollen joints decrease from 5 to 2, the number of tender joints from 10 to 0, the CRP from 47 to 25, the ESR from 40 to 12, the BASDAI score from 6.5 to 3.1, the night pain experienced by the patient from 34 mm to 4 mm, the general pain 33 mm to 12 mm, the disease activity from 41 mm to 23 mm.

No serious side effects or infections occur during the treatment. Except for the slight tiredness and mild muscle cramps, the patient experiences no other side effects.

### EXAMPLE 3

### Capsules with 4-[(4-methyl-1-piperazin-1-ylmethyl)-N-[4-methyl-3-[4-(3-pyridinyl)-2-pyrimidinyl]amino]phenyl]benzamide Monomethanesulfonate, β-Crystal Form

Capsules containing 119.5 mg of the compound named in the title (═SALT I) corresponding to 100 mg of COMPOUND I (free base) as active substance are prepared in the following composition:

Composition

| | | |
|---|---|---|
| SALT I | 119.5 | mg |
| Avicel | 200 | mg |
| PVPPXL | 15 | mg |
| Aerosil | 2 | mg |
| Magnesium stearate | 1.5 | mg |
| | 338.0 | mg |

The capsules are prepared by mixing the components and filling the mixture into hard gelatin capsules, size 1.

1-13. (canceled)

14. A method of treating humans suffering from spondylarthropathies which comprises administering to said human in need of such a treatment an effective dose of 4-(4-methylpiperazin-1-ylmethyl)-N-[4-methyl-3-(4-pyridin-3-yl)pyrimidin-2-ylamino)phenyl]-benzamide of the formula I 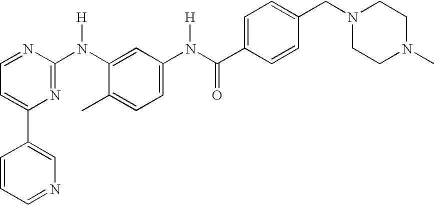
or a pharmaceutically acceptable salt thereof effective against spondylarthropathies.

15. The method according to claim 14 wherein the 4-(4-methylpiperazin-1-ylmethyl)-N-[4-methyl-3-(4-pyridin-3-yl)pyrimidin-2-ylamino)phenyl]-benzamide of the formula I is in the form of a Pharmaceutically acceptable acid addition salt.

16. The method according to claim 15 wherein the 4-(4-methylpiperazin-1-ylmethyl)-N-[4-methyl-3-(4-pyridin-3-yl)pyrimidin-2-ylamino)phenyl]-benzamide of formula I is in the form of the monomethanesulfonate salt.

17. The method according to claim 16 wherein the monomethanesulfonate salt of the Compound of formula I is administered at a daily dose corresponding to 100 to 1000 mg of the compound of formula I free base.

18. The method according to claim 14 wherein 4-(4-methylpiperazin-1-ylmethyl)-N-[4-methyl-3-(4-pyridin-3-yl)pyrimidin-2-ylamino)phenyl]-benzamide of the formula I or a pharmaceutically acceptable salt thereof is administered at a daily dose corresponding to 100 to 1000 mg of the Compound of formula I free base.

19. The method for according to claim 14 wherein the administration is once daily for a period exceeding 3 months.

20. The method according to claim 14 wherein the spondylarthropathy is the disease of a patient which fulfils the criteria for spondylarthorpathy as determined by the ESSG criteria, the AMOR criteria or both, ankylosing spondylitis, psoriatic arthropathy, undifferentiated spondyloarthropathy, reactive arthritis, Reiters syndrome, arthropathy associated with inflammatory bowl diseases, SAPHO syndrome or Morbus Bechet.

21. The method according to claim 14 wherein the spondylarthropathy is sporiatic arthritis.,

22. The method according to claim 14 wherein the spondylarthropathy is ankylosing spondylitis.

23. The method according to claim 14 wherein the spondylarthropathy is the disease of a patient which fulfils the criteria for spondylarthorpathy as determined by the ESSG criteria, the AMOR criteria or both.

24. A method of treating mammals suffering from spondylarthropathy which comprises administering to said mammal in need of such a treatment a pharmaceutical composition comprising:
(a) a dose, effective against spondylarthropathy, of 4-(4-methylpiperazin-1-ylmethyl)-N-[4-methyl-3-(4-pyridin-3-yl)pyrimidin-2-ylamino)phenyl]-benzamide of the formula I or a pharmaceutically acceptable salt thereof and
(b) a therapeutically effective amount of a disease modifying arthritis rheumatoid drugs abbreviated as DMARDs.

25. The method according to claim 24 wherein the combination partners are present in synergistically effective amount.

26. A commercial package comprising a combination which comprises (a) 4-(4-methylpiperazin-1-ylmethyl)-N-[4-methyl-3-(4-pyridin-3-yl)pyrimidin-2-yl-amino)phenyl]-benzamide or a pharmaceutically acceptable salt thereof and (b) a therapeutic agent selected from the disease modifying arthritis rheumatoid drugs together with instructions for use in the treatment of spondylarthropathy.
